(19) United States
(12) Patent Application Publication
(10) Pub. No.: US 2014/0274804 A1
(43) Pub. Date: Sep. 18, 2014

(54) ORGANIC MOLECULE SENSOR FOR DETECTING, DIFFERENTIATING, AND MEASURING ORGANIC COMPOUNDS

(71) Applicants: David Glynn Thomas, Wembley Downs (AU); Rachel Ellen Mohler, Emeryville, CA (US); Karen Ann Synowiec, Martinez, CA (US); Elaine Mae Bild, Berkeley, CA (US)

(72) Inventors: David Glynn Thomas, Wembley Downs (AU); Rachel Ellen Mohler, Emeryville, CA (US); Karen Ann Synowiec, Martinez, CA (US); Elaine Mae Bild, Berkeley, CA (US)

(73) Assignee: CHEVRON U.S.A. INC., San Ramon, CA (US)

(21) Appl. No.: 14/209,573

(22) Filed: Mar. 13, 2014

Related U.S. Application Data
(60) Provisional application No. 61/784,558, filed on Mar. 14, 2013.

(51) Int. Cl. G01N 33/18; G01N 33/00

(52) U.S. Cl. CPC G01N 33/1833; G01N 33/0047
USPC 506/15; 422/82.01; 422/98

(57) ABSTRACT

A sensor assembly is disclosed for detecting, speciating, and measuring a concentration of organic analytes in a fluid stream. The assembly includes a housing for containing elements of the sensor assembly; a sensor array configured to detect and measure the concentration of the organic analytes in the fluid sample and produce an electrical output signal indicative of a type and concentration of the organic analytes detected; an inlet channel through which a sample is drawn into the housing and into contact with the sensor array; an outlet channel through which the sample is expelled from the housing; and a sampler located within the housing for drawing the fluid sample into the housing via the inlet channel and expelling the sample via the outlet channel.

The present application for patent claims the benefit of U.S. provisional patent application bearing Ser. No. 61/784,558, filed on Mar. 14, 2013, which is incorporated by reference in its entirety.

### FIELD OF THE INVENTION

The invention relates to a sensor assembly for detecting, identifying, and measuring organic molecules such as hydrocarbons, particularly in a gas, crude, or water.

### BACKGROUND OF THE INVENTION

Hydrocarbons in water are rarely, if ever, measured in the aqueous phase. Frequently, the hydrocarbons are stripped from the aqueous phase to the vapour phase and then measured. Traditionally, the hydrocarbon in water samples are analysed on lab grade gas chromatographs. These devices are large, can consume significant quantities of carrier gas (such as hydrogen or helium), require a significant period of time to analyse the sample (typically of the order of 10 minutes), and require specialist knowledge to operate. Other methods for analysing hydrocarbons in water include portable field instruments (gas chromatographs or spectrometers), field based meters (such as Flame Ionisation Detectors (FIDs) and Photoionisation detectors (PIDs)), and screening kits that are geared toward a semi-quantitative or qualitative assessment of hydrocarbon content.

FIDs and PIDs have been used for analysis of hydrocarbon analytes during environmental site investigation and characterisation work in the US and around the world. The technology within both units has improved over the last 15 to 20 years since their introduction for field-based monitoring. However, these improvements have typically been with regard to reducing the cost and the overall size of the unit. The principle mechanism for the operation of both instruments has remained largely unchanged and therefore modern versions of these instruments retain their underlying limitations.

FIDs require a flame to measure combustible organics. FIDs require a fuel source to keep the flame lit and the flame must remain lit for it to work, therefore FIDs are sensitive to conditions that will extinguish the flame (moisture, dust, low oxygen environments etc). While FIDs are well suited for measuring combustible organic materials in air, they are less effective at measuring compounds without carbon-carbon bonds or organics with heteroatoms (e.g. chlorinated solvents).

PIDs require a lamp that emits photons at an energy sufficient to ionise various organic materials. In order for the organics to be detected by the device, the applied energy of the UV lamp must exceed the ionisation potential of the target chemical. PIDs are not able to ionise and detect some compounds such as carbon dioxide and methane.

Both of these devices have further limitations in that both instruments report the concentration of total organics. These devices do not have the ability to speciate or differentiate analytes in a sample. Each measurement outputs only one value.

At present there is nothing available that is ideally suited for active environmental monitoring applications e.g. rapid response time of the order of seconds, qualitative identification of hydrocarbon type, and accurate quantification for a broad range of analytes.

### SUMMARY OF THE INVENTION

The invention relates to a sensor assembly to detect and quantify organic molecules, such as hydrocarbons, in gas or liquids, such as natural gas, natural gas condensates, air, crude oil, refined petroleum gas or liquids, and water including connate water, condensed water and water containing hydrate inhibitor.

The sensor assembly includes a number of components including a sensor housing having a flow channel defined by an inlet, a sensor array, and an outlet. The sensor housing will also include a pump to draw a sample through the inlet, over the sensor array, and to expel the sample through the outlet.

The sensor array is based on the differential sorption properties measured using a surface acoustic wave (SAW) sensor array, a chemiresistor array, or a combination of the two.

Accordingly, in one aspect of the invention there is provided a sensor assembly for detecting, speciating, and measuring a concentration of organic analytes in a fluid stream, the assembly including: a housing for containing elements of the sensor assembly; a sensor array including a chemiresistor sensor array, an acoustics wave sensor array, or a combination thereof; an inlet channel through which a sample is drawn into the housing and into contact with the sensor array; an outlet channel through which the sample is expelled from the housing; and a sampler located within the housing for drawing the fluid sample into the housing via the inlet channel and expelling the sample via the outlet channel; wherein the sensor array is configured to detect and measure the concentration of the organic analytes and produce an electrical output signal indicative of a type and concentration of the organic analytes detected. Preferably the organic analytes are hydrocarbon analytes.

An advantage of the invention is that is that it provides an integrated sensor assembly that can be placed at a sampling location and left to run independently. Due to this arrangement, the sensor assembly has a small footprint in comparison with currently available downhole instrumentation. Typically, the volumetric size of the sensor is about 12 U.S. fl oz. This is significantly smaller that current state of the art hand held FID and PID field instruments.

The sensor assembly includes within the housing the essential components required for the sensor assembly to function and therefore is easy to install and operate. This provides flexibility in the installation of the sensor assembly, for example the sensor assembly can be rapidly deployed temporarily in a downhole without having to install ancillary equipment to accompany the sensor. Similarly, the sensor may be deployed semi-permanently or permanently, for example on a wellhead platform or processing plant.

Another advantage of this sensor assembly is that it permits the detection of organic analytes that would not otherwise be detectable by FIDs or PIDs. In an embodiment the sensor assembly is able to identify and quantify organic analytes with a carbon chain length of 3 to 40 atoms in water and gases (such as in the air). Preferably the organic analytes have a carbon chain length of 4 to 30 atoms.

Advantageously, the sensor assembly is able to provide an accurate and uniform response to concentration independent of the types of organic compounds in the sample. In certain embodiments the sensor assembly is able to detect methane (whether dissolved in a liquid such as water, or present in a gas such as the air) and other carbon containing compounds that cannot be readily ionised and thus detected using PIDs.

Advantageously, in certain embodiments the sensor is able to detect and quantify alkanes and alkenes including non-combustible or low-combustible compounds such as chlorinated alkane and alkene species including: vinyl chloride, dichloroethane, dichloroethene, trichloroethane, trichloroethene, tetrachloroethene, and other compounds that cannot be readily combusted and thus detected with FIDs.

In certain embodiments, the sensor is also able to differentiate between mixtures of organic compounds including pure (or near pure) methane, fresh gasoline, weathered gasoline, diesel, crude oil, and chlorinated alkanes and alkenes (e.g. differentiate between vinyl chloride and trichloroethene).

In an embodiment the sensor assembly will be used to detect, speciate, and measure a concentration of organic analytes in crude oil and natural gas reservoirs. Accordingly, in this embodiment the sensor assembly is configured to operate at temperatures of from about 60° C. to about 300° C. and under pressures of from about 2000 psi to about 8000 psi.

Another advantage of the invention resides in the use of chemiresistor and acoustic wave sensor arrays which allow the detection and speciation of organic analytes at a quantification limit of about 0.1 parts per million. The use of these sensors also permits continuous or near continuous measurements to be made, and provides for a rapid response time after exposure to a fluid sample of the order of seconds, such as less than one second. The sensor also requires a low sample volume and low volumetric flow rate of the order of millilitres per minute.

In an embodiment the sensor array includes a plurality of sensors each having a thiol layer to interact with the organic analytes. Preferably, the plurality of sensors includes at least two sensors having different thiol layers with different interaction strengths with the organic analytes. More preferably, the plurality of sensors includes at least three sensors having different thiol layers with different interaction strengths with the organic analytes, such that the plurality of sensors includes: a first sensor having a first thiol layer, the first thiol layer having a strong interaction strength with a first analyte, a second sensor having a second thiol layer, the second thiol layer having a weak interaction strength with the first analyte, and a third sensor having a third thiol layer, the third thiol layer having an interaction strength with the first analyte that is between the strong interaction strength and the weak interaction strength.

In an embodiment the sensor assembly further includes a power source located within the housing for powering the sampler and/or sensing component.

In an embodiment the sensor assembly further includes a processor located within the housing, the processor configured to: receive the electrical output signal from the sensor array; and apply principal component analysis to determine the type and concentration of the organic analytes detected. Alternatively, the sensor assembly further includes a processor located within the housing, the processor being configured to store as data the signal indicative of the type and concentration of the organic analytes detected. This data may either be raw data from the sensor array, or the processor may be configured to analyse the data to determine the type and concentration of the organic analytes detected. The data may then be downloaded on retrieval of the sensor assembly, or the data may be transmitted to a location remote from the sensor assembly.

In an embodiment the sensor assembly further includes a transmitter located within the housing for communicating to a remote location either the raw data from the sensor array or the analysed data including the type and concentration of the organic analytes detected.

In an embodiment the sensor array is a sensor array selected from the group consisting of a surface acoustic wave sensor array, a chemiresistor sensor array, or a combination of both.

In an embodiment the sensor array includes both a chemiresistor sensor array and an acoustics wave sensor array. Preferably the sensor array consists of both a chemiresistor sensor array and an acoustics wave sensor array.

The sensor housing may also include membrane interfaces and/or filters.

In an embodiment the sensor assembly is a gas phase sensor assembly. In an alternative embodiment the sensor is a liquid phase sensor assembly. In yet a further alternative embodiment, the sensor is both a gas and liquid phase sensor.

The sensor will be suitable for use in a range of applications, for example in: environmental monitoring for site assessment, characterisation and remediation of potential or known contaminated sites (e.g. sites with soil, sediment, or groundwater that is impacted or potentially impacted by organic and inorganic compounds); monitoring or identifying potential or known hydrocarbon leaks from buried or submerged pipelines, flow lines, transfer stations, or other hydrocarbon bearing structure; monitoring, identification, or investigation of buried or submerged hydrocarbon seeps for the purpose of exploration or effects related to shale gas or geosequestration activities; or mounting within a submerged tethered remote operated vehicle, untethered automatic operated vehicle, or marine glider.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1 is a block diagram of an embodiment of the invention.

### DETAILED DESCRIPTION OF THE EMBODIMENTS

The invention relates to a sensor assembly unit for the detection and quantification of organic analytes, such as hydrocarbons, in gas or liquids, such as natural gas, natural gas condensates, air, crude oil, refined petroleum gas or liquids, and water including connate water, condensed water and water containing hydrate inhibitor.

The sensor assembly is a module that has a housing that that incorporates the necessary components such that the sensor assembly has a small footprint and can quickly and easily be installed in a desired location, such as in an oil or gas reservoir, a downhole, a wellhead platform, or in a processing plant. It has been found that a sensor assembly including a sensor array chosen from a surface acoustic wave sensor, a chemiresistor, or both, is particularly well suited to the detection of hydrocarbon analytes.

Surface acoustic wave sensors are a class of microelectromechanical systems that rely on modulation of surface waves to detect, identify, and quantify hydrocarbon analytes in a fluid sample. Surface acoustic wave sensors use the piezoelectric effect in their operation. Surface acoustic wave sensors use an input interdigitated transducer (IDT) to convert an electrical signal into an acoustic wave. The transmitter IDT has a thin surface layer of a molecule such as an oligomer, polymer, or other organic molecule applied thereto. A known electrical signal is applied to the transmitter IDT to produce a known acoustic wave. A sorption interaction between an analyte and the surface layer can alters the transmitted acoustic wave. A receiver IDT converts the transmitted acoustic wave back into an electrical signal. The input electrical signal and the output electrical signal are then compared. Any significant difference will likely be the result of the interaction of the analyte and the surface layer on the transmitter IDT. Different analytes have different effects on the transmitted acoustic wave. An array of sensors can be constructed using a wide variety of organic molecules as the thin surface layer, such as different polymers, that differ in their responses to various analytes.

Chemiresistor sensors are made from conductive nanoparticles coated in a monolayer of a molecule such as an oligomer, polymer, or other organic molecule. The response of the sensor to a chemical is measured as a change in the resistance of the sensor. For a chemiresistor sensor, upon exposure to an analyte the analyte diffuses into the molecule and the molecule swells, which causes the dispersed conductive nanoparticles to move further apart from each other, causing the resistance of the sensor to increase. Different analytes have different effects on the resistance of the sensor. An array of polymer composite sensors can be constructed from a wide variety of organic molecules, such as different polymers, that differ in their responses to various analytes.

The sensor arrays of the invention may include a plurality of surface acoustic wave sensors, a plurality of chemiresistor sensors, or a combination of both surface acoustic wave sensors and chemiresistor sensors. At least some of the individual sensors in the array will be coated with different thiol molecules than other sensors. When the sensor array is exposed to a fluid (whether gas or liquid) containing either a single hydrocarbon analyte or a mixture of hydrocarbon analytes, each individual coated sensor device responds in a different manner due to a different interaction with the analyte (on account of the sensors possessing different thiol layers). As an example, certain thiol molecules will react strongly to aromatic compounds such as benzene and toluene, while other thiol molecules will react strongly to aliphatic compounds such as hexane.

Given the various responses of either a surface acoustic wave or chemiresistor sensor array to various analytes, samples can be classified, identified and quantified by using statistical methods, such as principle component analysis (PCA). This allows the types and concentrations of various hydrocarbon analytes in a sample to be determined.

Thiol molecules have been found to be particularly useful in the detection, identification, and quantification of hydrocarbon analytes. For this reason, the acoustic wave sensor arrays and chemiresistor sensor arrays of the present invention include a sensor layer that has been functionalised with thiol molecules. By selecting thiol molecules with a varied range of responses, it is possible to have an array of sensors that can detect, identify, and quantify a multitude of different hydrocarbon analytes.

It has been found that thiol layers that are formed from a thiol selected from the group consisting of substituted or unsubstituted: alkanethiol, alkenethiol, alkynethiol, arylthiol, heteroalkanethiol, heteroalkenethiol, heteroalkynethiol, or heteroarylthiol, are particularly useful in both acoustic wave sensor arrays and chemiresistor sensor arrays for the detection of a hydrocarbon analyte.

Additionally, thiol molecules that contain between 2 and 30 carbon atoms, preferably 4 to 20 carbon atoms, and even more preferably between 5 and 15 carbon atoms, are found to be particularly advantageous. Similarly, thiol molecules with a chain length of between 2 and 30 atoms, preferably 4 and 20 atoms, and even more preferably 5 and 15 atoms are found to be particularly advantageous.

Furthermore, particularly useful thiol molecules include those that are terminated at one end with a thiol, and at another end with a functional group selected from the group consisting of: carboxyl, carboxylate, hydroxyl, aldehyde, carbonyl, haloformyl, ester, peroxy, methoxy, amine, amide, aldimine, azide, cyanate, isocyanate, nitrile, isonotrile, nitrosooxy, nitro, nitroso, fluoride, chloride, bromide, iodide, thiol, sulfide, disulfide, sulfinyl, sulfonyl, sulfino, sulfo, thiocyanate, isothiocyanate, carbonothioyl, phosphino, phosphono, phosphate; or a fused or unfused substituted or unsubstituted 3 to 6 membered heterocyclic or aryl ring. The thiol portion of the molecule bonds with the metal surface of the surface acoustic wave sensor array or form a monolayer around the gold nanoparticles of the chemiresistor sensor array. Another end of the thiol molecule, having a functional group selected from above, is free to interact with the hydrocarbon analytes.

FIG. 1 provides an illustration of an embodiment of a sensor assembly 100. The sensor assembly 100 includes a sensor housing 102 having an inlet 104 and an outlet 106 and a flow path 108 between the inlet 104 and the outlet 106. A sampler (which in this case is a pump) 110 is mounted within the sensor housing 102. The pump 110 draws a fluid sample containing hydrocarbon analytes 112 through the inlet 104 and inside the sensor housing 102. The outlet of the pump 110 feeds the fluid sample to a sensor array 114. The analyte in the fluid sample 112 interacts with the surface of the sensors in the sensor array 114 to produce an output signal that is indicative of the type and concentration of hydrocarbon analytes in the fluid sample 112. This output signal is then received by a processor 116 which records the signal and then applies an algorithm, such as PCA, to convert the signal into data that represents the types and concentrations of hydrocarbon analytes detected. The processor then logs the data. The analysed fluid sample 120 is then exits from the sensor and is expelled from the sensor housing 102 through the outlet 106. A power supply 118, such as a battery, is used to supply power to the various components of the sensor assembly 100 that require power, such as the pump 110, the sensor array 114, and the processor 116. The sensor assembly 100 can then be retrieved and the data can be downloaded and analysed using if required.

As previously discussed, the sensor array 114 may be a surface acoustic wave sensor array, a chemiresistor sensor array, or a combination of both. By ‘array’ it is meant that the sensor array includes a plurality of sensors. In this particular embodiment the sensor array 114 includes a plurality of sensors, each of the sensors being functionalised with a thiol layer, some of the sensors having different thiol layers to other sensors.

While FIG. 1 relates to an embodiment in which a liquid fluid sample is analysed, it is intended that the sensor assembly can also be used to detect, identify, and quantify the presence of hydrocarbon analytes in a gas stream. In this situation the pump 110 may be replaced with another device to drive a gaseous fluid through the system such as a fan or blower.

In another alternative arrangement, the sensor assembly may be situated in a fluid stream (whether gas or liquid) and the sampler is an intake structure that feeds a fluid sample to the sensor. In this situation, the fluid pressure of the fluid stream is sufficient to drive the fluid sample through the inlet and the intake structure, over the sensor array, and then out through the outlet.

In another alternative arrangement, the processor communicates with a transmitter to transmit the information.

In a further alternative embodiment the sensor assembly includes a viewable display. In this alternative embodiment, the processor is configured to analyse the data from the sensor array to provide as an output the types and concentrations of hydrocarbon analytes present in the sample, and to transmit this information to the display to display the information.

In yet a further alternative embodiment other analytical tools can be used to interpret the raw data from the sensor array.

In an embodiment, the output signal from the sensor array may be processed by a computer, or a control system with a computer, and displayed as an output on a user interface. A notification device may be provided, which generates a notification that includes information relating to the type and concentrations of the various organic analytes in the sample. The control system may, for example, be a SCADA system, which provides system control and data acquisition. Where such instrumentation is provided, the data generated by the sensor assembly may be displayed locally in the vicinity of the sensor assembly. Alternatively or in addition, the data may be provided to the sensor assembly for display on a user interface and storage in memory.

In an embodiment the sensor assembly includes at least one computational device, which may be a microprocessor, a microcontroller, a programmable logical device or other suitable device. Instructions and data to control operation of the sensor assembly may be stored in a memory which is in data communication with, or forms part of, the computational device. Typically, the sensor assembly includes both volatile and non-volatile memory and may include more than one of each type of memory. The instructions and data for controlling operation of the sensor assembly may be stored on a computer readable medium from which they are loaded into the memory. Instructions and data may be conveyed to and from the sensor assembly by means of a data signal in a transmission channel. Examples of such transmission channels include network connections, the internet or an intranet and wireless communication channels.

It will be understood that the invention disclosed and defined in this specification extends to all alternative combinations of two or more of the individual features mentioned or evident from the text or drawings. All of these different combinations constitute various alternative aspects of the invention.

1. A sensor assembly for detecting, speciating, and measuring a concentration of organic analytes in a fluid stream, the assembly including:
a housing for containing elements of the sensor assembly;
a sensor array configured to detect and measure the concentration of the organic analytes in the fluid sample and produce an electrical output signal indicative of a type and concentration of the organic analytes detected;
an inlet channel through which a sample is drawn into the housing and into contact with the sensor array;
an outlet channel through which the sample is expelled from the housing; and
a sampler located within the housing for drawing the fluid sample into the housing via the inlet channel and expelling the sample via the outlet channel.

2. The sensor assembly of claim 1, wherein the sensor array includes a plurality of sensors each having a thiol layer to interact with the organic analytes.

3. The sensor assembly of claim 2, wherein the plurality of sensors includes at least two sensors having different thiol layers with different interaction strengths with the organic analytes.

4. The sensor assembly of claim 3, wherein the plurality of sensors includes at least three sensors having different thiol layers with different interaction strengths with the organic analytes, such that the plurality of sensors includes:
a first sensor having a first thiol layer, the first thiol layer having a strong interaction strength with a first organic analyte,
a second sensor having a second thiol layer, the second thiol layer having a weak interaction strength with the first organic analyte, and
a third sensor having a third thiol layer, the third thiol layer having an interaction strength with the first organic analyte that is between the strong interaction strength and the weak interaction strength.

5. The sensor assembly of claim 2 wherein each of the thiol layers includes a thiol selected from the group consisting of substituted or unsubstituted: alkanethiol, alkenethiol, alkynethiol, arylthiol, heteroalkanethiol, heteroalkenethiol, heteroalkynethiol, or heteroarylthiol.

6. The sensor assembly of claim 5, wherein the thiol contains between 2 and 20 carbon atoms.

7. The sensor assembly of claim 6, wherein the thiol contains between 5 and 15 carbon atoms.

8. The sensor assembly of claim 5, wherein the thiol is terminated at one end with a functional group selected from the group consisting of: carboxyl, carboxylate, hydroxyl, aldehyde, carbonyl, haloformyl, ester, peroxy, methoxy, amine, amide, aldimine, azide, cyanate, isocyanate, nitrile, isonotrile, nitrosooxy, nitro, nitroso, fluoride, chloride, bromide, iodide, thiol, sulfide, disulfide, sulfinyl, sulfonyl, sulfino, sulfo, thiocyanate, isothiocyanate, carbonothioyl, phosphino, phosphono, phosphate; or a fused or unfused substituted or unsubstituted 3 to 6 membered heterocyclic or aryl ring.

9. The sensor assembly of claim 1, further including a power source located within the housing for powering the sampler and/or sensing component.

10. The sensor assembly of claim 1, further including a processor located within the housing, the processor configured to:
receive the electrical output signal from the sensor array; and
apply principal component analysis to determine the type and concentration of the organic analytes detected.

11. The sensor assembly of claim 1, further including a transmitter located within the housing for communicating the type and concentration of the organic analytes detected to a remote location.

12. The sensor assembly of claim 1, wherein the sensor housing includes a viewable display that displays the type and concentration of the organic analytes detected.

13. The sensor assembly of claim 1, wherein the sensor array is configured to detect hydrocarbon analytes in a gas at a quantification limit of about 1 μg/m³ and/or detect organic analytes in a liquid at a quantification limit of about 1 μg/L.

14. The sensor assembly of claim 1, wherein the sensor assembly is configured to operate at temperatures of from about 60° C. to about 300° C.

15. The sensor assembly of claim 1, wherein the sensor assembly is configured to operate at pressures of from about 2000 psi to about 8000 psi.

16. The sensor assembly of claim 1, wherein the sensor array is a surface acoustic wave sensor array, a chemiresistor sensor array, or a combination of both.

17. The sensor assembly of claim 16, the sensor array including both a chemiresistor sensor array and an acoustics wave sensor array.

18. The sensor assembly of claim 1, wherein the organic analytes are organic molecules with a carbon chain length of 3 to 40 atoms.

19. The sensor assembly of claim 18, wherein the organic analytes are organic molecules with a carbon chain length of 4 to 30 atoms.
